(19) United States
(12) Patent Application Publication
(10) Pub. No.: US 2013/0282121 A1
(43) Pub. Date: Oct. 24, 2013

(54) SPINAL FACET AUGMENTATION IMPLANT AND METHOD

(71) Applicant: Ann Prewett, Bloomfield Hills, MI (US)

(72) Inventor: Ann Prewett, Bloomfield Hills, MI (US)

(21) Appl. No.: 13/848,873

(22) Filed: Mar. 22, 2013

Related U.S. Application Data
(60) Provisional application No. 61/614,327, filed on Mar. 22, 2012.

(51) Int. Cl. A61F 2/30

(52) U.S. Cl. CPC A61F 2/30756
USPC 623/17.12

(57) ABSTRACT

A spinal facet joint implant suitable for minimally invasive implantation and capable of transitioning from a compact first configuration to an expanded second configuration is made of a swellable and compressible fluid absorbing polymer. The implant is dimensioned and configured, while in the first configuration, to fit within the lumen of a surgical needle. In the expanded, second configuration, the implant exerts pressure within the facet joint which helps keep the facet joint from collapsing and anchors the implant in place. A spinal facet joint implant herein may also be dimensioned and configured to occupy a portion of the capsule of a facet joint. A spinal facet joint implant for treating inflammation made from a fluid absorbing polymer absorbs and sequesters inflammatory agents as it absorbs synovial fluid in situ. Methods of making and implanting a spinal facet implant are provided.

### FIELD OF THE INVENTION

Augmentation or cushioning between the facet joints for treatment of pain related conditions of the spine.

### DESCRIPTION OF THE RELATED ART

Physical discomfort from degenerative conditions of the spine facet joints affects a large segment of the population. In normal operation, a facet is not loaded but can experience some sheer forces as the joint moves. As the facets age and degenerate they can swell, become inflamed and the cartilaginous “endplate” like structure of the facet degrades. The facets start to rub on each other and produce pain.

Conservative treatment may include rest, physical therapy, bracing, anti-inflammatory medications, analgesics, local anesthetic blocks and steroid injections. Surgical intervention may be required including posterior dynamic stabilization and fusion. These invasive procedures may be associated with loss of mobility and can lead to complications.

### SUMMARY OF THE INVENTION

In embodiments, an injectable facet joint cartilage replacement implant for treatment of a damaged facet joint includes a fluid retaining hydrogel material dimensioned and configured for passage through a small diameter needle into a space within a facet joint, wherein upon absorption of fluid, the hydrogel material assumes a three dimensional conformation corresponding to the space between the facets to augment the function of damaged facet cartilage.

In embodiments, a spinal facet implant suitable for minimally invasive implantation includes an implant body having first and second configurations, the first configuration having reduced volume compared to the second configuration, the first configuration being dimensioned and configured to fit within a lumen of a surgical needle, the second configuration dimensioned and configured to fit between a superior articular face of a facet joint and an inferior articular face of the facet joint, the second configuration further dimensioned and configured to exert sufficient pressure against the superior articular face of the facet joint and the inferior articular face of the facet joint to keep said faces separated, wherein the implant transitions from the first configuration to the second configuration by swelling.

In embodiments, a self-anchoring spinal facet implant includes an implant body having first and second configurations, the first configuration having reduced volume compared to the second configuration, the second configuration dimensioned and configured to fit between a superior articular face of a facet joint and an inferior articular face of the facet joint, the second configuration further dimensioned and configured to exert sufficient pressure against the superior articular face of the facet joint and the inferior articular face of the facet joint to keep said faces separated and to keep the implant body anchored within the facet joint without need of a separate anchor device, wherein the implant transitions from the first configuration to the second configuration by swelling.

In embodiments, an implant for treating inflammation associated with an irritated facet joint includes a fluid absorbing injectable hydrogel material dimensioned and configured for passage through a small diameter needle into the synovial or capsular tissue surrounding the facet joint that absorbs inflammatory agents in the facet joint.

In embodiments, a method for treating inflammation associated with an irritated facet joint includes positioning an at least partially dehydrated fluid absorbing polymer in the facet joint and allowing the fluid absorbing polymer to absorb and sequester fluids containing inflammatory agents from within the facet joint, thus reducing exposure of the inflammatory agents to the irritated facet joint.

### BRIEF DESCRIPTION OF THE DRAWINGS

FIG. 1 is a cross-sectional view of a human spinal facet joint.

FIG. 2 is a cross-sectional view illustrating a human spinal facet joint with the tip if a needle inserted therein for delivering a spinal facet implant in a reduced volume configuration. Also illustrated in the facet joint is an embodiment of a spinal facet implant in a reduced volume configuration in accordance with the disclosure herein.

FIG. 3 is a cross-sectional view illustrating a human spinal facet joint containing an embodiment of a spinal facet implant in an enlarged volume configuration in accordance with the disclosure herein.

FIG. 4 is a cross-sectional view illustrating a human spinal facet joint with the tip if a needle inserted therein for delivering a fluid absorbing injectable hydrogel material in an at least partially dehydrated configuration. Also illustrated in the facet joint is an embodiment of a spinal facet implant in a reduced volume configuration in accordance with the disclosure herein.

FIG. 5 is a dimensional view of an embodiment of a spinal facet implant in a reduced volume configuration in accordance with the disclosure herein.

FIG. 6 is a dimensional view of an embodiment of a concave spinal facet implant in an enlarged volume configuration in accordance with the disclosure herein.

FIG. 7 is a dimensional view of an embodiment of a spinal facet implant in a reduced volume configuration in accordance with the disclosure herein.

FIG. 8 is a dimensional view of an embodiment of a convex spinal facet implant in an enlarged volume configuration in accordance with the disclosure herein.

FIG. 9 is a dimensional view of an embodiment of a spinal facet implant in a reduced volume configuration in accordance with the disclosure herein.

FIG. 10 is a dimensional view of an embodiment of a curved spinal facet implant in an enlarged volume configuration in accordance with the disclosure herein.

FIG. 11 is a dimensional view of an embodiment of a spinal facet implant in a reduced volume configuration in accordance with the disclosure herein.

FIG. 12 is a dimensional view of an embodiment of a flat spinal facet implant in an enlarged volume configuration in accordance with the disclosure herein.

FIG. 13 is a cross-sectional view illustrating a human spinal facet joint containing an embodiment of a spinal facet implant in an enlarged volume configuration positioned within the capsule of the facet joint in accordance with the disclosure herein.

FIG. 14 is a dimensional view of an embodiment of a spinal facet implant in an enlarged volume configuration and containing a support member in accordance with the disclosure herein.

FIG. 15 is a dimensional view of an embodiment of a spinal facet implant in an enlarged volume configuration and containing a support member which, in part, extends outside the periphery of the body of the implant in accordance with the disclosure herein.

### DETAILED DESCRIPTION

A swellable, resilient, self-retaining facet augmentation implant is provided that includes a swellable polymeric medium, said polymeric medium being dispersed throughout the implant, the implant being dimensioned and configured to fit between the articulating surfaces of a facet joint. In embodiments, the implant has a first configuration of comparatively reduced size, volume and/or mass such that it can be inserted into the patient in a minimally invasive manner. The swellable polymeric medium may be a fluid absorbing polymer, e.g., a hydrogel. The swellable polymeric medium may also be a substantially non-fluid absorbing elastic polymer. In embodiments, the implant is capable of expanding from a compact, substantially dehydrated first configuration to an expanded hydrated configuration, also referred to herein as the second configuration. In embodiments, the implant is configured to transform from a first configuration to a second configuration, the first configuration having a smaller cross-section than the second configuration. In embodiments, the implant is capable of undergoing anisotropic expansion from the first configuration to the second configuration. In embodiments, the implant is capable of undergoing isotropic expansion from the first configuration to the second configuration.

In embodiments, the implant includes an interiorly disposed support member. In embodiments, at least a portion of the interiorly disposed support member may extend beyond the periphery of the implant. In embodiments the support member is made of flexible fibers. The flexible fibers may be made, e.g., from natural or synthetic polymers or metal. In embodiments, the support member is fabric selected from the group consisting of mesh, woven fabric and nonwoven fabric made of flexible fibers. In embodiments, the support member is a braided three-dimensional support member made of flexible fibers. In embodiments, the interstices of the braided three-dimensional support member are filled with the polymeric medium. In embodiments, the support member is a flexible foil made from metal or a polymer. The support member may be constructed from relatively durable materials including, but not limited to, metal foil, plastic foil, metal fibers, polymeric fibers of materials such as polycarbonate, polyethylene, polypropylene, polystyrene, polyethylene terephthalate, polyamide, polyurethane, polyurea, polysulfone, polyvinyl chloride, acrylic and methacrylic polymers, expanded polytetrafluoroethylene (Goretex®), ethylene tetrafluoroethylene, graphite, etc. Polyester mesh made of Dacron® (commercially available from E.I. du Pont de Nemours and Company) or nylon are especially suitable. These materials can be used either alone, or in a composite form in combination with elastomers or hydrogels. Especially advantageous are mesh, woven, non-woven, perforated, or porous formats of these materials which will allow solid anchoring in the implant. Alternatively, the support member may be exteriorly disposed, e.g., a jacket which surrounds all or part of the implant. In embodiments, at least a portion of the implant includes a wear reducing surface to prevent degradation of the implant due to friction caused by articulation of the facets. The wear reducing surface can be a clearly defined separate layer such as a sheath or patch, or it can be an integral layer which has no clearly defined boundary between the material which makes up the body of the implant and the wear reducing surface. The wear reducing surface serves to protect the interiorly disposed cushiony material which makes up the body of the implant and provides a smooth, durable contact surface which reduces friction and consequent wear of the implant and/or bone. In embodiments, a radiopaque material may be included in or around the implant.

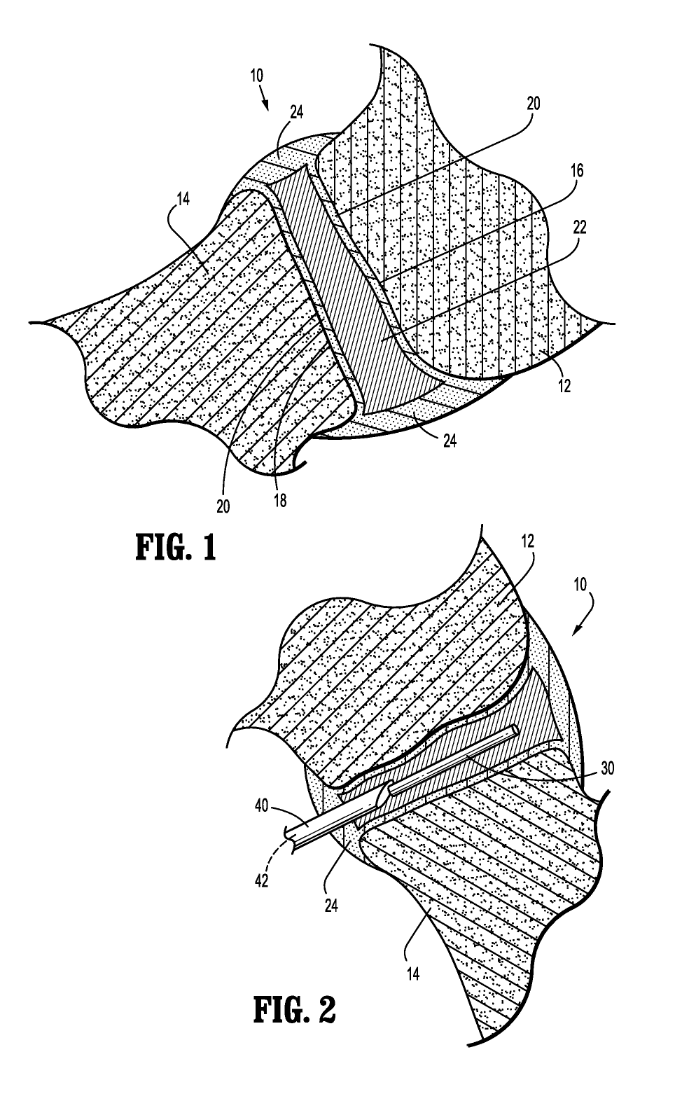

FIG. 1 illustrates a cross-sectional view of a human facet joint 10 which includes a superior articular facet 12 and an inferior articular facet 14. The superior articular facet 12 has a superior articular face 16 and the inferior articular facet 14 has an inferior articular face 18. The facet joint 10 is a synovial joint defined by the opposing superior and inferior articular faces 16 and 18 with cartilage 20 between them and a capsule 24 further defining a substantially water tight structure. Synovial fluid 22 is contained inside the joint 10 by the capsule 24 and keeps the joint faces 16 and 18 lubricated. The ends of the bone articular facets 12 and 14 that make up the synovial facet joint 10 are normally covered with the articular, hyaline cartilage 20 that allows the bony faces 16, and 18 to slide against one another, providing the flexibility that allows the movement of vertebral bodies relative to one another.

A facet augmentation implant according to the present disclosure provides a minimally invasive, tissue-sparing alternative to more invasive techniques. FIG. 2 illustrates a cross-sectional view of a human spinal facet joint 10 having a surgical needle 40 (partially shown) inserted therein through the capsule 24. A compact embodiment of a facet implant 30 is injected through the lumen 42 of the needle 40 into the joint cavity and positioned within the facet joint. In embodiments, the facet augmentation implant is compact for insertion (first configuration) and may have a variety of shapes In embodiments, an implant made of a fluid absorbing polymer can be dehydrated to provide a compact insertion shape. As it absorbs fluid, e.g., in an in vivo setting, it expands to achieve a desired final shape for in-dwelling function where it serves to lubricate the facet and act as a spacer. It is contemplated that the in-dwelling shape of the implant should fit between two facets and augment the space between them without interfering with adjacent spinal architecture. For example, in embodiments, the insertion shape of the implant has a rod or cylindrical shape ranging, e.g., from about 2 mm to about 10 mm in length and from about 0.1 mm to about 3 mm in diameter. In embodiments, the diameter may range from about 0.4 mm to about 1 mm. Other convenient insertion shapes are contemplated such as pyramidal, ellipsoidal, accordion, taco, bullet and the like. In embodiments, the implant expands isotropically or anisotropically to form a substantially flat disc or polygon from about 0.2 mm thick to about 1 mm thick. Indeed, or any flattened shape that accommodates the facet joints is contemplated.

In embodiments, the facet implant according to the present disclosure expands within the facet joint upon absorption of fluids until the dimensions of the cavity formed within the joint serve to restrict further expansion of the implant. In this manner, the implant mechanically serves to distract the facets 12 and 14 and prevent them from rubbing against each other as may be the case when the facet joint is damaged or deteriorating. Once in place, the implant expands, depending on the method of compaction as discussed herein, either isotopically or anistropically, to its expanded configuration which, due to the swellability and resiliency of the polymer, at least partially conforms to the topography of the facet joint space between adjacent facets 12, 14. As a result, an facet implant according to the present disclosure provides a cushiony custom fit for the implant that, along with the frictional anchoring engagement discussed below, avoids the need for rigid, traumatic attachments to the vertebral bone. In embodiments, the unconstricted inherent maximum volume of the implant is greater than the space contained within the facet joint cavity. The tendency of the hydrogel facet implant to absorb fluid until maximum inherent volume is reached serves to create a distraction force against the opposing faces 16, 18. In addition, the expansion force mechanically engages the faces 16, 18 and exerts sufficient pressure against them to anchor the implant in place and prevent unwanted movement or dislodging of the implant.

An advantage of the cushiony nature of the implant allows it to be used in patients with osteoporosis. Typically, such patients have brittle bones which may break under heavy loads. Rigid implants of the prior art would be contraindicated in these patients since they are unyielding and can concentrate too much load on osteoporotic bone, thereby increasing the risk of fracture or breakage. The present implant reduces or eliminates the propensity of weakened, osteoporotic bone to fracture and/or break.

Fluid absorbing polymers are well-suited for manufacturing a swellable, resilient facet implant in accordance with the present disclosure. Suitable fluid absorbing polymers include synthetic polymers such as poly(ethylene glycol), poly(ethylene oxide), partially or fully hydrolyzed poly(vinyl alcohol), poly(vinylpyrrolidone), poly(ethyloxazoline), poly(ethylene oxide)-co-poly(propylene oxide) block copolymers (poloxamers and meroxapols), poloxamines, carboxymethyl cellulose, and hydroxyalkylated celluloses such as hydroxyethyl cellulose and methylhydroxypropyl cellulose, and natural polymers such as polypeptides, polysaccharides or carbohydrates such as Ficoll™, polysucrose, hyaluronic acid, dextran, heparan sulfate, chondroitin sulfate, heparin, or alginate, and proteins such as gelatin, collagen, albumin, or ovalbumin or copolymers or blends thereof. As used herein, “celluloses” includes cellulose and derivatives of the types described above; “dextran” includes dextran and similar derivatives thereof. Examples of materials that can be used to form a hydrogel include modified alginates. Alginate is a carbohydrate polymer isolated from seaweed, which can be crosslinked to form a hydrogel by exposure to a divalent cation such as calcium. Alginate is ionically crosslinked in the presence of divalent cations, in water, at room temperature, to form a hydrogel matrix. Modified alginate derivatives may be synthesized which have an improved ability to form hydrogels.

Additionally, polysaccharides which gel by exposure to monovalent cations, including bacterial polysaccharides, such as gellan gum, and plant polysaccharides, such as carrageenans, may be crosslinked to form a hydrogel using methods analogous to those available for the crosslinking of alginates described above. Polysaccharides which gel in the presence of monovalent cations form hydrogels upon exposure, for example, to a solution comprising physiological levels of sodium. Hydrogel precursor solutions also may be osmotically adjusted with a nonion, such as mannitol, and then injected to form a gel.

Other polymeric hydrogel precursors include polyethylene oxide-polypropylene glycol block copolymers such as Pluronics™ or Tetronics™, which may be crosslinked by hydrogen bonding and/or by a temperature change. Other materials which may be utilized include proteins such as fibrin, collagen and gelatin. Polymer mixtures also may be utilized. For example, a mixture of polyethylene oxide and polyacrylic acid which gels by hydrogen bonding upon mixing may be utilized. In embodiments, a mixture of a 5% w/w solution of polyacrylic acid with a 5% w/w polyethylene oxide (polyethylene glycol, polyoxyethylene) 100,000 can be combined to form a gel over the course of time, e.g., as quickly as within a few seconds.

Water soluble polymers with charged side groups may be crosslinked by reacting the polymer with an aqueous solution containing ions of the opposite charge, either cations if the polymer has acidic side groups or anions if the polymer has basic side groups. Examples of cations for cross-linking of the polymers with acidic side groups to form a hydrogel are monovalent cations such as sodium, divalent cations such as calcium, and multivalent cations such as copper, calcium, aluminum, magnesium, strontium, barium, and tin, and di-, tri- or tetra-functional organic cations such as alkylammonium salts. Aqueous solutions of the salts of these cations are added to the polymers to form soft, highly swollen hydrogels and membranes. The higher the concentration of cation, or the higher the valence, the greater the degree of cross-linking of the polymer. Additionally, the polymers may be crosslinked enzymatically, e.g., fibrin with thrombin. The polymers can be covalently crosslinked as well through the addition of ethylene diamine, NBS or a host of crosslinking agents routinely to react with amino, nitrile, urethane and carboxylic functional groups found on the polymer chain.

Suitable ionically crosslinkable groups include phenols, amines, imines, amides, carboxylic acids, sulfonic acids and phosphate groups. Aliphatic hydroxy groups are not considered to be reactive groups for the chemistry disclosed herein. Negatively charged groups, such as carboxylate, sulfonate and phosphate ions, can be crosslinked with cations such as calcium ions. The crosslinking of alginate with calcium ions is an example of this type of ionic crosslinking. Positively charged groups, such as ammonium ions, can be crosslinked with negatively charged ions such as carboxylate, sulfonate and phosphate ions. Preferably, the negatively charged ions contain more than one carboxylate, sulfonate or phosphate group.

Anions for cross-linking of the polymers to form a hydrogel are monovalent, divalent or trivalent anions such as low molecular weight dicarboxylic acids, for example, terepthalic acid, sulfate ions and carbonate ions. Aqueous solutions of the salts of these anions are added to the polymers to form soft, highly swollen hydrogels and membranes, as described with respect to cations.

A variety of polycations can be used to complex and thereby stabilize the polymer hydrogel into a semi-permeable surface membrane. Examples of materials that can be used include polymers having basic reactive groups such as amine or imine groups, having a preferred molecular weight between 3,000 and 100,000, such as polyethylenimine and polylysine. These are commercially available. One polycation is poly(L-lysine); examples of synthetic polyamines are: polyethyleneimine, poly(vinylamine), and poly(allyl amine). There are also natural polycations such as the polysaccharide, chitosan.

In preferred embodiments, the facet implant is made of a hydrogel. Prior to coagulation, the liquid form of a suitable hydrogel is used to form the expanded configuration as it would be in the hydrated state. The hydrogel is then coagulated to form the implant in an expanded configuration. The implant is then dehydrated to a xerogel state which reduces the volume of the implant to the reduced configuration. Many hydrogel polymers behave in a similar manner, which is to say they can be deformed, frozen into a deformed shape and they can maintain that shape indefinitely or until, e.g., a temperature change causes the polymer to “relax” into the shape originally held prior to freezing. This property is often referred to as shape memory or frozen deformation by those skilled in the art.

The temperature at which frozen deformation occurs is referred to as the glass transition temperature or T_{g}. At T_{g} several polymer properties such as density, entropy and elasticity may sharply change. Many polymers can be mixed with agents that can have a drastic effect on a polymer T_{g}. Polymers which absorb fluid are of particular interest and water is the preferred T_{g} altering agent. Hydrogels which contain less than about five percent water may be considered dehydrated or xerogels. The T_{g} of a xerogel will change as it absorbs fluids containing water. Once the T_{g} becomes lower than ambient the now partially hydrated hydrogel becomes pliant and may be elastically deformed. If the polymer is held in a state of elastic deformation while the T_{g} is raised above ambient the polymer will maintain the deformed state indefinitely. This can be accomplished by either lowering the ambient temperature (freezing) or by returning the polymer to its xerogel state thus raising the T_{g}.

Using this method, hydrogel articles may be produced with vastly differing xerogel shapes compared to hydrated shapes. This is especially useful in cases such as medical implants where, in delivering a prosthesis into the human body, every care should be taken to reduce trauma to the patient. An implant which is shaped as disc, for instance, is re-shaped into a rod in order to facilitate minimally invasive implantation. Alternatively, a portion of the implant can be compressed as compared to another portion of the implant. Indeed, various frozen shapes may be utilized to facilitate implantation and situation of the implant. Once the implant is indwelling and has absorbed bodily fluids it will substantially return to the expanded shape and maintain that shape indefinitely. As used herein, “substantially” is intended to mean any of “approximately”, “nearly” or “precisely.”

A preferred polymer configuration includes two polymer phases of different hydrophilicity, the less hydrophilic phase having higher content of hydrophobic groups and more hydrophilic phase having higher content of hydrophilic groups. The less hydrophilic phase is preferably crystalline and more hydrophilic phase is preferably amorphous, as can be established from X-ray diffraction.

Advantageous hydrophobic groups are pendant nitrile substituents in 1,3 positions on a polymethylene backbone, such as poly(acrylonitrile) or poly(methacrylonitrile). The hydrophilic phase may preferably contain a high concentration of ionic groups. Preferred hydrophilic groups are derivatives of acrylic acid and/or methacrylic acid including salts, acrylamidine, N-substituted acrylamidine, acrylamide and N-substituted acryl amide, as well as various combinations thereof. A particularly preferred combination contains approximately two thirds acrylic acid and its salts (on molar basis), the rest being a combination of plain and N-substituted acrylamides and acrylamidines.

At least one polymeric component is preferably a multiblock copolymer with alternating sequences of hydrophilic and hydrophobic groups. Such sequences are usually capable of separating into two polymer phases and form strong physically crosslinked hydrogels. Such multiblock copolymers can be, for example, products of hydrolysis or aminolysis of polyacrylonitrile or polymethacrylonitrile and copolymers thereof. For convenience, polymers and copolymers having at least about 80 molar % of acrylonitrile and/or methacrylonitrile units in their composition may be referred to as “PAN”. Hydrolysis and aminolysis of PAN and products thereof are described, for example, in U.S. Pat. Nos. 4,107,121; 4,331,783; 4,337,327; 4,369,294; 4,370,451; 4,379,874; 4,420,589; 4,943,618, and 5,252,692, each being incorporated herein by reference in their respective entireties.

A preferred fluid absorbing polymer for the facet implant is a synthetic composite of a cellular (or domain) type with continuous phase formed by a hydrophobic polymer or a hydrophilic polymer with low to medium water content forming a “closed cell” spongy structure that provides a composite with good strength and shape stability. Examples of suitable polymers are polyurethanes, polyureas, PAN, and highly crystalline multiblock acrylic and methacrylic copolymers. The polymer should be sufficiently permeable to water. More preferably, the continuous phase is formed by a strong hydrophilic polymer with sufficient permeability for water but impermeable to high-molecular solutes. Examples of such polymers are highly crystalline hydrogels based on segmented polyurethanes, polyvinylalcohol or multiblock acrylonitrile copolymers with derivatives of acrylic acid. Typically, suitable polymers for the continuous phase in cellular composites have a water content in fully hydrated state between about 60% by weight and about 90% by weight, preferably between about 65% and about 85% by weight.

The second component of the fluid absorbing polymer may be a highly hydrophilic polymer of high enough molecular weight to prevent permeation of the hydrophilic polymer through the continuous phase. This component is contained inside the matrix of the continuous phase. The entrapped hydrophilic polymers (the so-called “soft block”) may be high-molecular weight water-soluble polymers, associative water-soluble polymers or highly swellable hydrogels containing, in a fully hydrated state, an amount of hydration which is preferably at least about 5% greater than the hydrophobic component. For example, the second component hydrated to at least about 65% when the first component is hydrated to about 60%. In other embodiments, e.g., from the second component could be fully hydrated at from about 95% of water and up to about 99.8% of water. Such hydrogels are very weak mechanically. However, it may not matter in composites where such polymers' role is generation of osmotic pressure rather than load-bearing, with e.g., compression strength in full hydration in the range of about 0.01 MN/m² or lower.

A system with closed cells (or domains) containing highly swellable or water-soluble polymers can form composites with very high swelling pressure as needed for the facet implant function. Examples of suitable hydrophilic polymers are high-molecular weight polyacrylamide, polyacrylic acid, polyvinylpyrrolidone, polyethyleneoxide, copolymers of ethyleneoxide and propyleneoxide or hyaluronic acid; covalently crosslinked hydrogels such as hydrophilic esters or amides of polyacrylic or polymethacrylic acids; and physically crosslinked hydrogels, such as hydrolyzates or aminolysates of PAN.

Particularly suitable are associative water-soluble polymers capable of forming very highly viscous solutions or even soft physical gels. Preferred are associative polymers containing negatively charged groups, such as carboxylates, sulpho-groups, phosphate groups or sulfate groups. Particularly preferred are associative polymers formed by hydrolysis and/or aminolysis of PAN to high but finite conversions that leave a certain number of nitrile groups (typically, between about 5 and 50 molar %) unreacted.

Preferred fluid absorbing polymer composites have both a continuous phase and a dispersed phase formed by different products of hydrolysis or aminolysis of PAN. In this case, both components are compatible and their hydrophobic blocks can participate in the same crystalline domains. This improves anchorage of the more hydrophilic component and prevents its extraction or disassociation. The size of more hydrophilic domains may vary widely, from nanometers to millimeters, preferably from tens of nanometers to microns.

The ratio between the continuous discrete phase (i.e., between more hydrophobic and more hydrophilic components may vary from about 1:1 to about 1:100 on a dry weight basis, and a preferred ratio ranges from about 1:2 to about 1:20. Examples of compositions and implants are described in U.S. Pat. Nos. 6,264,695 and 6,726,721, both of which are incorporated herein by reference in their entireties. A method of making the fluid absorbing polymer composite is described in U.S. Pat. No. 6,232,406, herein incorporated by reference in its entirety.

Examples of particularly suitable hydrogel forming copolymers are prepared by a partial alkaline hydrolysis of polyacrylonitrile (“HPAN”) in the presence of sodium thiocyanate (NaSCN). The resulting hydrolysis product is a multi-block acrylic copolymer, containing alternating hydrophilic and hydrophobic blocks. Hydrophilic blocks contain acrylic acid, acrylamidine, and acrylamide. In embodiments, for example, a PAN hydrolysate polymer (referred to herein HPAN I) (46±1% conversion of hydrolysis) having the following composition: acrylonitrile units ˜53-55%, acrylic acid units ˜22-24%, acrylamide units ˜17-19%, acrylamidine units ˜4-6%, as determined by ¹³C NMR, is dissolved in a suitable solvent such as a ˜55% solution of sodium thiocyanate in water to form a viscous solution. The viscous solution is poured into a porous mold having, e.g., an hourglass shaped cavity. The solution can then be solvent cast, e.g., by solvent exchange (e.g., water for NaSCN). The pores should be sufficiently small as to not permit the polymer to diffuse or leak out of the mold. In another form, the hydrogel used to make a facet implant is obtained by reacting an aquagel of PAN, formed by dissolving the polymer in an aqueous solvating solution such as high concentration of sodium thiocyanate. The resulted solution of PAN is thereupon coagulated through addition of a suitable aqueous solvent or water miscible solvent. The coagulum is further reacted in a hydrolyzing basic or acidic medium. The PAN aquagel can then be processed as a thermoplastic and molded to obtain the desired shape. These methods are described in U.S. Pat. No. 4,943,618 incorporated herein by reference in its entirety.

A more rigid fluid absorbing polymer may be another PAN hydrosylate polymer, referred to herein as HPAN II (28±1% conversion of hydrolysis), having the following composition: acrylonitrile units ˜71-73%, acrylic acid units ˜13-15%, acrylamide units ˜10-12%, acrylamidine units ˜2-4%, as determined by ¹³C NMR, dissolved in ˜55% NaSCN which can be solvent cast, washed, dried and cut to a suitable shape.

In embodiments, a method of making a swellable, resilient, facet implant includes providing a mold defining a cavity adapted and configured to approximate at least a portion of the space between two articulating surfaces of a facet joint, providing a liquid polymer, filling the mold with the liquid polymer and coagulating the liquid polymer to form a swellable, resilient, self-retaining facet augmentation implant dimensioned and configured to fit between two articulating surfaces of a facet joint. In embodiments, the liquid polymer is a fluid absorbing polymer. The fluid absorbing polymer can be a hydrogel. In embodiments, the method includes placing a support member into the mold prior to filling with liquid polymer. In embodiments, a facet implant may be manufactured by providing a support member such as a suitably shaped mesh or a three-dimensional braided support member of desired configuration and placing it in a mold. In embodiments, the support member is a braided three-dimensional member configured and dimensioned to have a shape consistent with the mold cavity. In embodiments, a support member, e.g., a braided three-dimensional support member, is placed within a mold cavity which has dimensions greater than the braided three-dimensional support member to allow the liquid fluid absorbing polymer to be absorbed into and saturate the braided three-dimensional support member and to encapsulate the braided three-dimensional support member with a layer of fluid absorbing polymer. A fluid absorbing liquid polymer is added to the mold and infuses into the interstices of the support member until the support member is preferably saturated. In embodiments, a gap, e.g., about 1 mm, is left between one or more sides of the support member and the walls of the mold. Fluid absorbing liquid polymer is allowed to fill the gap between the mold and the support member. As the support member absorbs fluid absorbing liquid polymer additional amounts of the fluid absorbing liquid polymer can be added. When the fluid absorbing polymer is cured or fixed, e.g., by solvent casting, ionic gelation, photo-polymerization and the like, it solidifies and creates a continuous matrix throughout the support member and also forms a layer surrounding and encapsulating the support member. In the case of solvent casting, the mold may be made of material which is impermeable to the fluid absorbing polymer but permeable to water. The mold is placed in a water bath to extract the solvent (e.g., sodium thiocyanate) which causes the polymer to coagulate. The mold may then be opened and any remaining solvent in the implant is extracted. If it is desired to leave one or more sides of the implant open to the support member, then the desired side(s) of the support member is placed up against the wall of the mold to prevent formation of a gap for the liquid fluid absorbing polymer to fill.

In embodiments, the fluid absorbing polymer is made to achieve a strong physical bond to the fibers of the support member by incorporating an initial treatment of the fibers of the member, either before or after the weaving or braiding process, with a relatively hydrophobic fluid absorbing polymer to create an encapsulating layer of the relatively hydrophobic fluid absorbing polymer. For example, a hydrogel such as HPAN II is applied to the fibers as a 10% solution by weight in a solvent (sodium thiocyanate 55% by weight in water) and then coagulated onto the fibers by solvent exchange with an aqueous solution such as water. As the polymer coagulates, it shrinks volumetrically around the fibers, causing a tight physical bond to the fibers. If desired, the treated support member is placed in a mold and a relatively more hydrophilic fluid absorbing polymer in the liquid state is added to create a cohesive continuous polymer matrix which surrounds the support member. For example, a 10% by weight HPAN I in a 55% by weight sodium thiocyanate solution, is added to the mold. The solvent from the HPAN I solution causes the outermost surface of the coagulated HPAN II layer surrounding the braided fibers to dissolve and allow commingling of the HPAN I and HPAN II hydrogel polymers at the surface interface which forms a strong adhesive bond when the HPAN I and commingled hydrogels are coagulated by solvent exchange. It should be understood that the support member is optional and that a mold may be filled without such a support member.

In embodiments, one or more tethers such as a string, suture, etc., are incorporated into the facet implant. The tether may be utilized in positioning or maintaining the position of the facet implant, or its components, during manufacture in molds, and after manufacture as a device for positioning and helping maintain the implant within a facet joint space. The tether may be simply placed in a central location within a support member prior to filling a mold with a liquid fluid absorbing polymer and is then present when the cavity is filled. Alternatively, a tether may be incorporated into the center of a mold when a liquid fluid absorbing polymer insert is coagulated. Alternatively, the tether may be attached directly to the support member at either an interior location or an exterior location if the support member extends out of the fluid absorbing body of the implant.

In embodiments, the facet augmentation implant is dehydrated to reduce the dimensions, volume and/or mass of the implant. In order to obtain a preferred rod-shape having an optimal cross-sectional shape for implantation a reduction in volume deformation is advantageously maintained radially, substantially parallel with a longitudinal axis. This may be accomplished by placing the implant within a radially collapsible member for exerting circumferential compression on an object, e.g., a facet implant, contained within the member. Suitable radially collapsible members include, e.g., a flexible sleeve such as a braided sock or tube, a flexible coil, iris diaphragm, collapsible loop, etc. In a preferred embodiment, the radially collapsible member is porous or semipermeable so that water, either as liquid or as vapor, passes through the member. The collapsible member may be made of an elastic material such as rubber or neoprene fabric which has been made porous by any technique known to those skilled in the art, or a woven or non-woven mesh or braid. The collapsible member may also be made of a flexible metal having sufficient porosity to allow water to exit from the implant. The collapsible member does, however, need to be stiff enough to be able to exert sufficient compressive force when tension is applied to compress the facet implant, i.e., it should not be so elastic that it deforms without being able to exert sufficient compressive force.

In operation, the radially collapsible member exerts substantially equilateral circumferential compression on the facet implant by substantially uniformly decreasing in diameter while contacting the implant. The preferred porous nature of the collapsible member allows water from the implant to escape into the surrounding environment so that the facet implant can become dehydrated. In embodiments, the sleeve radially collapsible member is stretched in length which causes the inner diameter to decrease, thus compressing the implant, including, e.g., a braided three-dimensional reinforcement member, into a desired implantation configuration. A more complete description of a suitable radial compression process is described in U.S. Pat. No. 7,806,934, herein incorporated by reference in its entirety. Other methods of reducing the profile of the implant include folding or rolling the implant into, e.g., bellows, a taco shape or a cigar shape.

The collapsible member is loaded in tension via any tensioning device known to one skilled in the art, e.g., a pneumatic cylinder, a hydraulic cylinder, springs, weights, pulleys, etc. The tension on the collapsible member can be precisely controlled by regulating the pressure within the tensioning device, translating into constant, controlled radial load on the implant. In the case of a sleeve collapsible member, once the implant is loaded into the collapsible member and the collapsible member is tensioned, three things occur: the implant dehydrates, the implant deforms, the collapsible member extends. By varying the tension on the collapsible member, the length of the implant can be extended, thereby decreasing the minor axis and height. This can also be controlled, to some extent, by the speed of dehydration (temperature, pressure and humidity), with longer dehydration time producing longer implant length and vise versa.

In embodiments, the majority of the dehydration process can occur at room temperature over an extended period of time (e.g., 18 to 36 hours). The facet implant can be monitored to determine the extent of dehydration and the time period adjusted accordingly. Relative humidity, air circulation, air pressure and room temperature should be controlled during this period. For example, conditions may be about 21° C. at 50% relative humidity under moderate airflow. Once the implant has reached <˜30% water content it may be forced dry at elevated temperature, e.g., from about 25° C. to about 105° C. for typically less than about 24 hours to rapidly remove remaining water. As above, the state of dehydration may be monitored to determine if greater or lesser amounts of time are needed. When the implant is substantially completely dehydrated, the implant is fairly rigid in its state of frozen deformation. Alternatively, a slight degree of hydration provides some flexibility to the implant. The less dehydrated, the more flexible. It is contemplated herein that “substantially dehydrated” preferably encompasses from about 12% or less, to about 30% water by weight of the implant.

Upon completion of forced dehydration, the implant is extremely stable in terms of shelf life, providing that it is kept dry. Even brief exposure to humidity during the sterilization process should not have significant effects. Temperatures above about 80° C. should be avoided for extended periods as this may bring the implant above its T_{g} if it has absorbed some small amount of water vapor.

Surface irregularities may be present on a dehydrated compressed implant which was compressed as described above by a radially collapsible member by virtue, e.g., of some extrusion of the hydrogel through pores or through interstitial spaces of the member. For example, a woven or non-woven collapsible sleeve may have interstitial spaces that allow hydrogel to extrude therein under compressive force. In addition, after radial compression, as described above, the dimensions of the implant may be different than the ultimate dimensions desired by the practitioner. Both of these instances can be remedied by post-compression thermoforming of the implant. In this aspect, a dehydrated, compressed implant is placed within a mold which may be advantageously pre-heated to about 70-150° C., but more preferably, closer to the melting point of the polymer, e.g., about 105° C. Care must be taken to avoid subjecting the implant to excess heat which causes the hydrogel to exceed its critical point, and thus causing permanent deformation of the implant. If the temperature is high, the implant must be quickly removed from the mold to avoid permanent deformation. The mold is machined to the exact desired final dimensions of the xerogel implant and essentially irons out surface roughness to a substantially smooth surface, which is less abrasive to surrounding tissue when implanted. If desired, and if the xerogel implant is compressed by a radially compressive member or by gas compression, but has not achieved, e.g., an ideal enough straight rod-like configuration, or if the ends are not sufficiently blunted or otherwise tapered, post-compression thermoforming may be utilized to fine tune the shape as well as remove any surface irregularities which may be present. Post-compression thermoforming may also be utilized to bend an implant to a desired configuration.

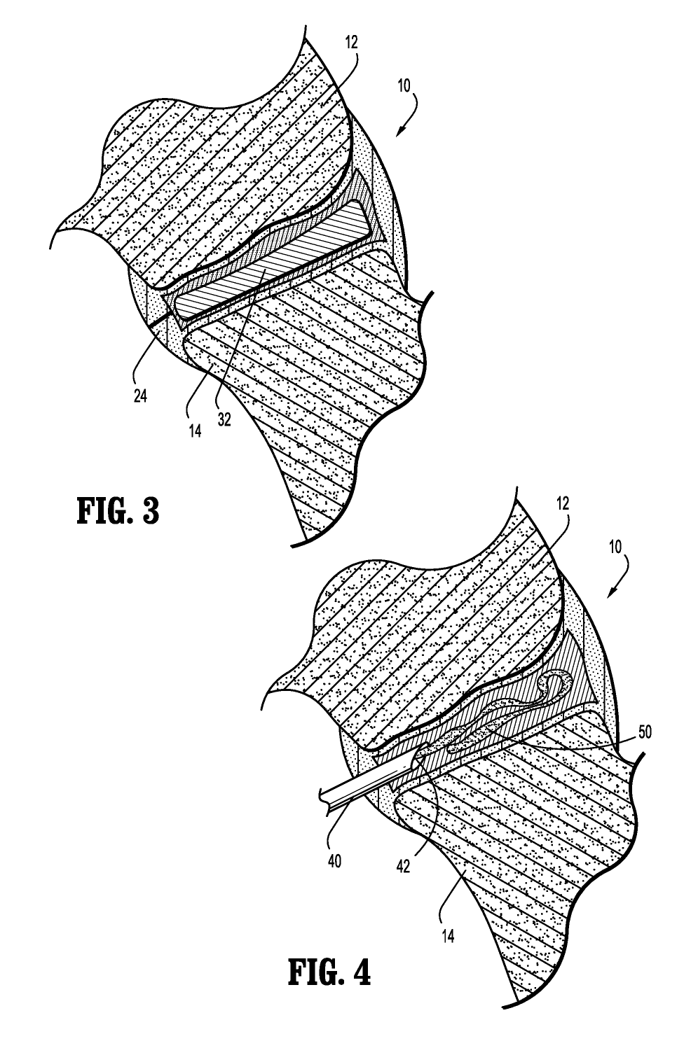

In embodiments, a facet implant according to the present disclosure is delivered within the facet capsule using a small gauge needle, e.g., a 17 or 18 gauge needle. The compact configuration, also referred to herein as a first configuration, has a reduced volume and/or mass as compared to the expanded configuration also referred to herein as the second configuration). Dehydration of the fluid absorbing polymer causes it to lose volume and/or mass. Conversely, absorption of fluid causes the fluid absorbing polymer (and the implant) to gain volume and/or mass. The compact configuration is dimensioned and configured to fit within the lumen of a surgical needle. Needles are routinely used in facet blocks and other procedures to deliver pain medication. It is contemplated that well-known techniques that are used to produce a facet block injection can be used to implant the compact facet augmentation implant. For example, the needle can be placed between the facets under fluoroscopic guidance, the implant is then pushed through the needle and, while in the space between facets, the implant expands to produce an in-dwelling space such as a disc or a polygon that will be dimensioned and configured to fit the space between the facet joints. FIG. 3 illustrates a cross-sectional view of a human spinal facet joint 10 containing an embodiment of a spinal facet implant 32 in an expanded configuration. An implant made of a fluid absorbing polymer such as a hydrogel gains mass and/or volume as it hydrates in vivo and provides a soft lubricious spacer between the superior and inferior articulating surfaces. Since the hole created in the capsule by the needle is narrow, expansion of the implant in the facet joint creates an implant cross-section larger than the hole which also serves to prevent the implant from being ejected through the hole.

The pathology of a degenerating facet joint is complex and results from a combination of physical and chemical assaults. Implants according to the present disclosure are especially well-suited to address both physical and chemical causative agents of facet joint degeneration. Use of minimally invasive techniques and a fluid absorbing hydrogel in accordance with the present disclosure provides localized procedures for efficaciously treating degenerative joint disease. In addition to physical degradation of the facet joint from frictional wear and tear, without wishing to be bound by any particular theory, the present inventors believe that pro-inflammatory molecules (inflammatory agents) within a joint capsule contribute to degeneration of and/or pain within the joint in at least one of the following ways: a) chemical sensitization of nerve fibrils contained within the collagenous ligaments of the joint capsule; b) chemical sensitization of nerve fibrils contained within the synovium; c) mediation of and/or direct degeneration of the hyaline articular surfaces; and d) chemical sensitization of nerve fibrils adjacent or in close proximity to a joint capsule. Continued irritation can result in chronic inflammation which may become a self sustaining process under these circumstances.

Inflammatory agents include cytokines, chemokines and complement system components. Examples include interferons, tumor necrosis factors (TNF), membrane attack complex (MAC), C3, C5a, Factor XII and other inflammatory peptides and proteins of a molecular weight typically between 300 daltons and 30000 daltons. Such inflammatory agents are well-known to those skilled in the art.

In addition to serving a mechanical function, a substantially dehydrated fluid absorbing facet implant according to present disclosure absorbs synovial fluid rich in inflammatory agents after implantation in a diseased and/or degenerating facet joint. In this manner, inflammatory agents are removed from the surrounding environment and sequestered in the implant, thereby reducing the deleterious effects of the inflammatory agents. FIG. 4 illustrates an embodiment of the invention wherein a substantially dehydrated fluid absorbing injectable hydrogel material 50 dimensioned and configured for passage through a small diameter needle 40 is injected into the synovial or capsular tissue surrounding the facet joint. As the hydrogel material 50 occupies the joint space, it absorbs bodily fluids that contain inflammatory agents while swelling to buttress and augment the function of cartilage in the joint space. Similarly, the embodiments shown in FIGS. 2 and 3 perform the same function, i.e., they can comprise a dehydrated fluid absorbing injectable hydrogel implant 30 which absorbs bodily fluid containing inflammatory agents to reduce the amount of the inflammatory agents floating freely in the joint space while expanding to the second configuration 32 which performs the mechanical function of keeping the opposing faces 14, 16 separated and lubricated by virtue of the lubricious surfaces of the implant. In embodiments, the hydrogel swells upon absorption of fluid and compresses by expression of fluid based upon load applied by said superior and inferior faces during normal movement thereby maintaining contact with the opposing faces 14, 16.

The implant may be manufactured by providing a mold that allows the implant to be cast, e.g., as a flat disc or other in-dwelling shape and coagulated. The implant is then reshaped into an insertion shape, e.g., rod shape, or others. Techniques for such reshaping are described, e.g., in U.S. Pat. No. 7,806,934, incorporated herein by reference in its entirety. Using such techniques, the implant may be shaped, dimensioned and configured to create a dehydrated elongated shape that can be introduced into an appropriate gauge needle. Upon fluid absorption, the implant can undergo anistropic change to form the flat disc.

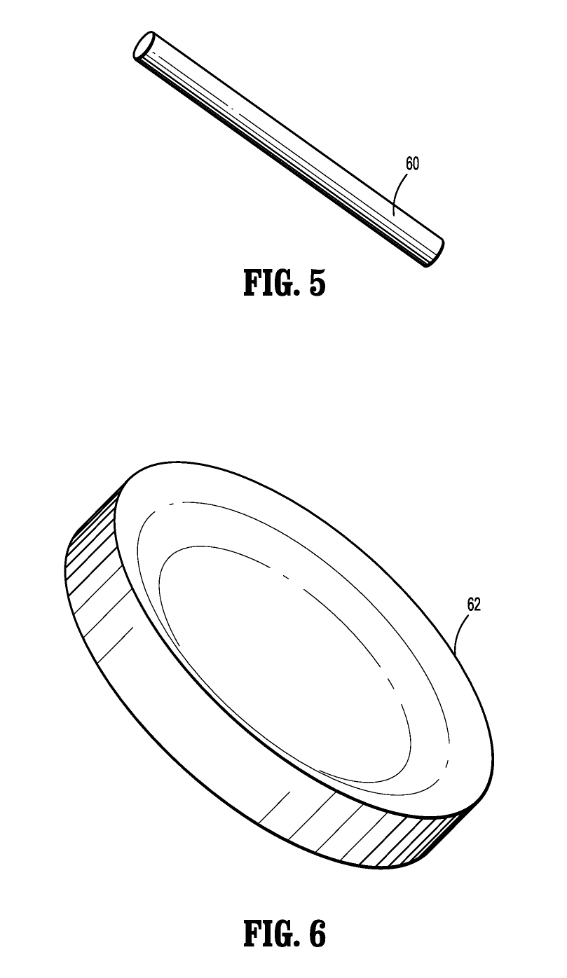

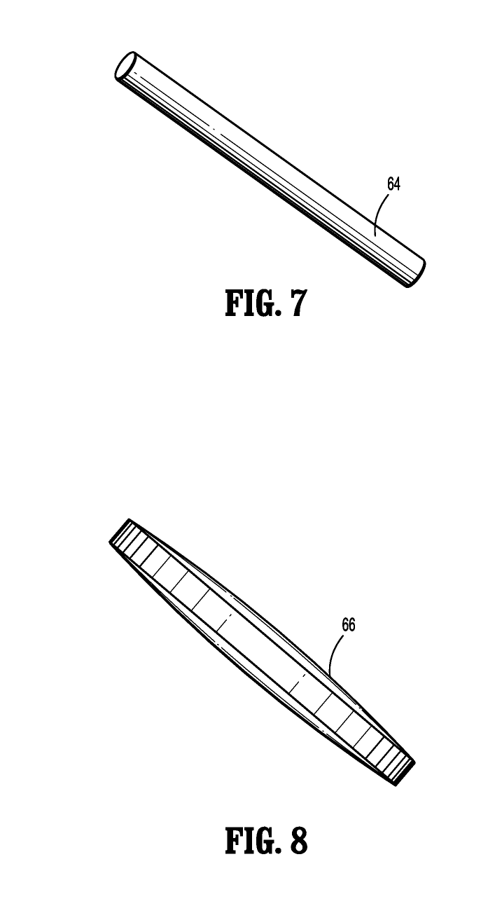

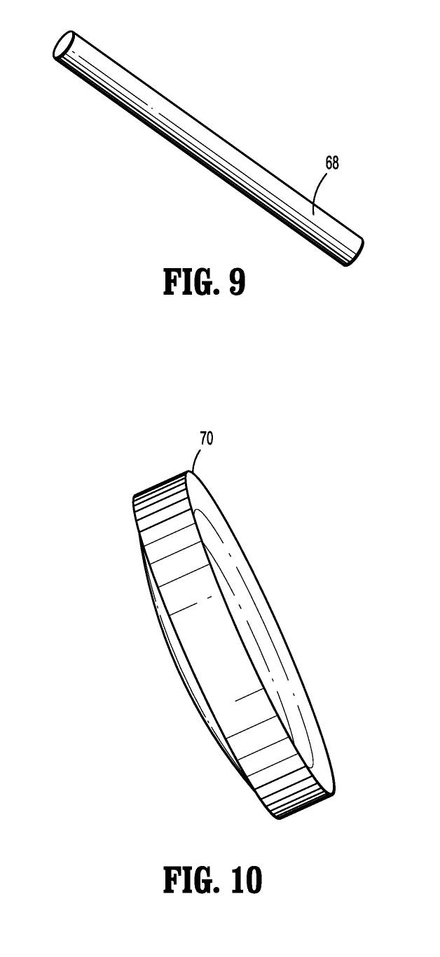

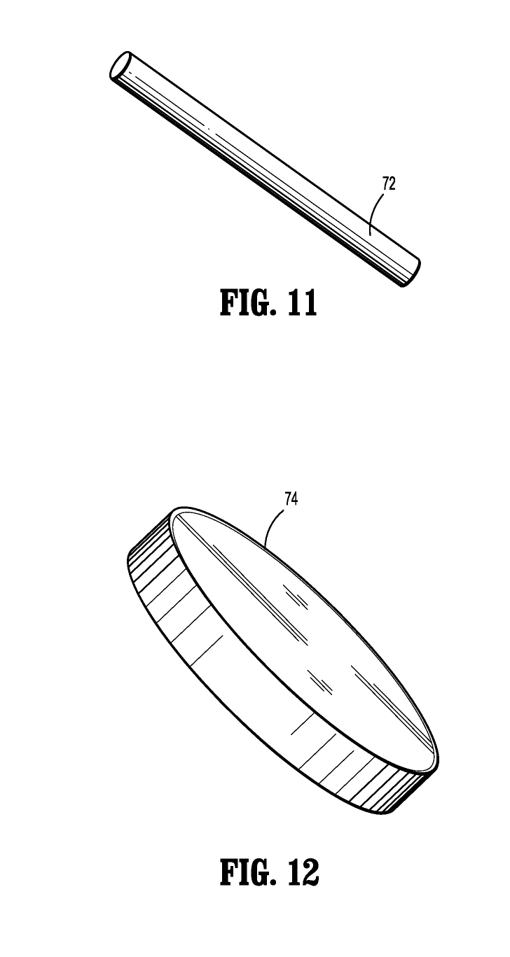

The indwelling shape of the implant can be predetermined to suit the needs of a particular facet joint. For example, as illustrated dimensionally in FIG. 5, a facet implant 60 in a first configuration is rod-shaped and anisotropically expands to a concave disc-shaped second configuration 62 as shown, e.g., in FIG. 6. One or both sides of the disc may have a concave aspect. As illustrated dimensionally in FIG. 7, a facet implant 64 in a first configuration is rod-shaped and anisotropically expands to a convex disc-shaped second configuration 66 as shown, e.g., in FIG. 8. One or both sides of the disc may have a convex aspect. As illustrated dimensionally in FIG. 9, a facet implant 68 in a first configuration is rod-shaped and anisotropically expands to a curved disc-shaped second configuration 70 as shown, e.g., in FIG. 10. As illustrated dimensionally in FIG. 11, a facet implant 72 in a first configuration is rod-shaped and anisotropically expands to a flat disc-shaped second configuration 74 as shown, e.g., in FIG. 12. One or both sides of the disc may have a convex aspect.

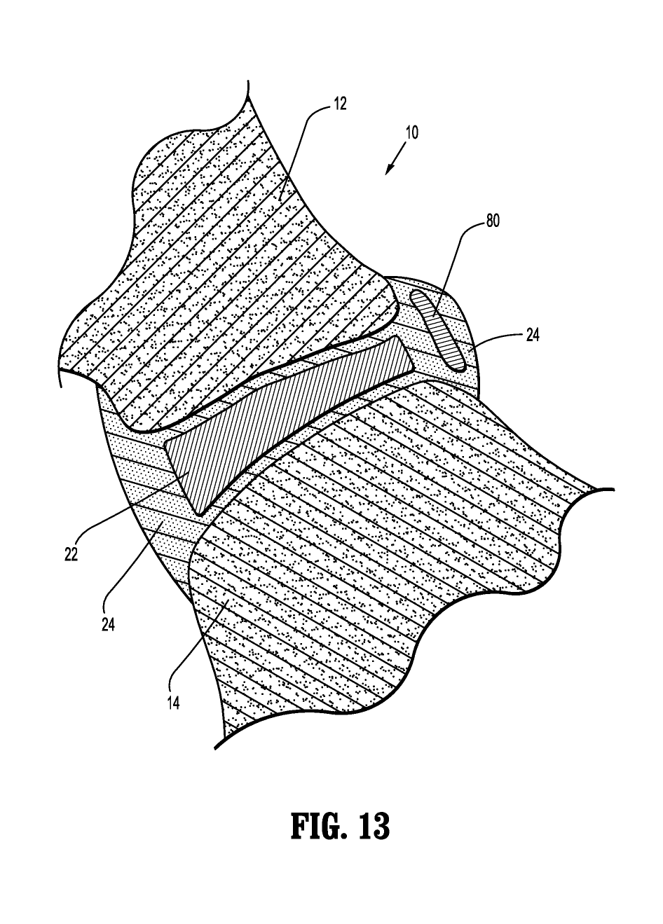

In embodiments, a facet implant according to the present disclosure may be dimensioned and configured for insertion into the capsule 24. As illustrated in cross-section in FIG. 13, a facet joint 10 contains a facet implant 80 in the capsule 24. Any embodiment of a facet implant described herein may be utilized for this purpose. The relative size of the first configuration and second configuration are adjusted to fit within the target location on the capsule.

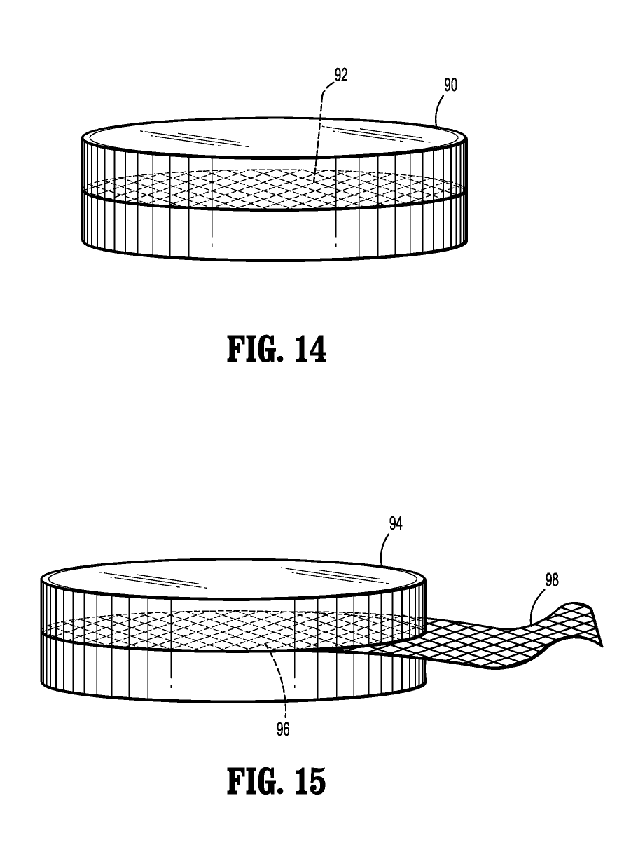

FIG. 14 illustrates a facet implant 90 in the second configuration which includes an interiorly disposed mesh support member 92. FIG. 15 illustrates a facet implant 94 in the second configuration which includes a mesh support member 96 that includes a portion 98 which extends out of the body of the implant 94.

In addition to serving a mechanical function, the implant can be used as a drug delivery platform. This can be accomplished in a variety of ways. For example, the dehydrated implant can come preloaded and as the implant hydrates and stabilizes, one or more medicinal agents are released into the surroundings to achieve a therapeutic effect. In embodiments, the implant is hydrated in vivo with a medicinal agent containing solution. The medicinal agent diffuses into the fluid absorbing polymer along with the fluid and remains locally concentrated for extended time periods. In embodiments, the hydrated implant swells and releases medicated fluid in response to load or sheer.

“Medicinal agent” is used in its broadest sense and it includes any substance or mixture of substances which may have any clinical use. It is to be understood that medicinal agent encompasses any drug, including hormones, antibodies, therapeutic peptides, etc., or a diagnostic agent such as a releasable dye which has no biological activity per se. Thus, in its broadest aspect, a method of delivery herein may be defined as the release of any substance for clinical use, which may or may not exhibit biological activity.

Examples of medicinal agents that can be used include anesthetics, anti-inflammatory agents, growth factors such as BMPs, antimicrobials, anticancer agents, analgesics, and radiopaque materials. Such medicinal agents are well-known to those skilled in the art. The medicinal agents may be in the form of dry substance in aqueous solution, in alcoholic solution or particles, microcrystals, microspheres or liposomes. An extensive recitation of various medicinal agents is disclosed in Goodman and Gilman, The Pharmacological Basis of Therapeutics, 10th ed. 2001, or Remington, The Science and Practice of Pharmacy, 21 ed. (2005). As used herein, the term “antimicrobial” is meant to encompass any pharmaceutically acceptable agent which is substantially toxic to a pathogen. Accordingly, “antimicrobial” includes antiseptics, antibacterials, antibiotics, antivirals, antifungals and the like. Radiopaque materials include releasable and non-releasable agents which render the implant visible in any known imaging technique such as X-ray radiographs, magnetic resonance imaging, computer assisted tomography and the like. The radiopaque material may be any conventional radiopaque material known in the art for allowing radiographic visualization of an implant, and may be, e.g., metal wire or flakes made from a biocompatible material, such as titanium, tantalum, stainless steel, or nitinol; or metallic salts (such as barium compounds).

Medicinal agents may be incorporated into a facet implant at various points in the manufacturing process. For example, a suitable medicinal agent can be mixed with a fluid absorbing liquid polymer before it is cured or fixed. Alternatively, a suitable medicinal agent may be dissolved into a solvent cast solution and then diffused into the hydrogel in accordance with normal kinetic principles. If the implant is then dehydrated, the medicinal agent collects in the interstices of the hydrogel and/or the braided three-dimensional reinforcement member.

A dehydrated facet implant according to the disclosure herein may be sterilized by any suitable conventional means, e.g., ethylene oxide, irradiation, etc. and packaged for distribution. A kit containing the sterilized implant and a package insert describing the facet implant, along with instructions is useful for medical practitioners.

In embodiments, the implant may contain a substance which allows the implant to be visualized in vivo using X-ray techniques, ultrasound or other visualization techniques. For example, the implant can be manufactured to contain particles of pt-ir wire that can be visualized via X-ray. In addition, a thin layer of fabric containing a pt it wire can be incorporated into implant. In this manner, the wire will be a marker for positioning and allowing visualization via X-ray after surgical placement to ensure proper fit.

It should be understood that the examples and embodiments of the invention provided herein are preferred embodiments. Various modifications may be made to these examples and embodiments without departing from the scope of the invention which is defined by the appended claims. For example, those skilled in the art may envision additional polymers and/or hydrogels which can be compacted and shaped according to the techniques described herein. Similarly, the shapes of the compacted and hydrated or expanded facet implant described herein are exemplary and any suitable compacted and/or expanded facet implant shape can be subjected to the techniques described herein to create an optimally shaped, substantially dehydrated facet implant for minimally invasive insertion into the facet joint. Those skilled in the art can envision additional radially collapsible members for exerting substantially uniform radial compression on the implant which are not set forth herein. In addition, process parameters such as temperature, humidity, pressure, time and concentration may be varied according to conventional techniques by those skilled in the art to optimize results.

What is claimed is:

1. A spinal facet implant suitable for minimally invasive implantation comprising an implant body having first and second configurations, the first configuration having reduced volume compared to the second configuration, the first configuration being dimensioned and configured to fit within a lumen of a surgical needle, the second configuration dimensioned and configured to fit between a superior articular face of a facet joint and an inferior articular face of the facet joint, the second configuration further dimensioned and configured to exert sufficient pressure against the superior articular face of the facet joint and the inferior articular face of the facet joint to keep said faces separated, wherein the implant transitions from the first configuration to the second configuration by swelling.

2. The spinal facet implant according to claim 1 wherein the first configuration is a rod shape.

3. The spinal facet implant according to claim 1 wherein the second configuration is a disc shape.

4. The spinal facet implant according to claim 1 wherein the implant body is made of a fluid absorbing polymer.

5. The spinal facet implant according to claim 4 wherein the fluid absorbing polymer transitions from the first configuration to the second configuration by absorption of bodily fluid.

6. The spinal facet implant according to claim 4 wherein the polymer swells upon absorption of fluid and compresses by expression of fluid based upon load applied by said superior and inferior faces.

7. The spinal facet implant according to claim 4 wherein the fluid absorbing polymer is a hydrogel.

8. The spinal facet implant according to claim 1 further comprising a support member disposed within the implant body.

9. The spinal facet implant according to claim 8 wherein the support member includes an exteriorly disposed portion extending out of the implant body, the exteriorly disposed portion being dimensioned and configured to be attached to surrounding tissue and/or bone.

10. The spinal facet implant according to claim 1 wherein the implant body gains mass when transitioning from the first configuration to the second configuration.

11. A self-anchoring spinal facet implant comprising an implant body having first and second configurations, the first configuration having reduced volume compared to the second configuration, the second configuration dimensioned and configured to fit between a superior articular face of a facet joint and an inferior articular face of the facet joint, the second configuration further dimensioned and configured to exert sufficient pressure against the superior articular face of the facet joint and the inferior articular face of the facet joint to keep said faces separated and to keep the implant body anchored within the facet joint without need of a dedicated anchor device, wherein the implant transitions from the first configuration to the second configuration by swelling.

12. The self-anchoring spinal facet implant according to claim 11 wherein the first configuration is a rod shape.

13. The self-anchoring spinal facet implant according to claim 11 wherein the second configuration is a disc shape.

14. The self-anchoring spinal facet implant according to claim 11 wherein the implant body is made of a fluid absorbing polymer.

15. The self-anchoring spinal facet implant according to claim 14 wherein the fluid absorbing polymer transitions from the first configuration to the second configuration by absorption of bodily fluid.

16. The self-anchoring spinal facet implant according to claim 14 wherein the polymer swells upon absorption of fluid and compresses by expression of fluid based upon load applied by said superior and inferior faces.

17. The self-anchoring spinal facet implant according to claim 14 wherein the fluid absorbing polymer is a hydrogel.

18. The self-anchoring spinal facet implant according to claim 11 further comprising a support member disposed within the implant body.

19. The self-anchoring spinal facet implant according to claim 18 wherein the support member includes an exteriorly disposed portion extending out of the implant body, the exteriorly disposed portion being dimensioned and configured to be attached to surrounding tissue and/or bone.

20. The self-anchoring spinal facet implant according to claim 11 wherein the implant body gains mass when transitioning from the first configuration to the second configuration.

21. A method of implanting a spinal facet implant into a spinal facet joint comprising:
providing a syringe having a surgical needle containing the spinal facet implant wherein the implant comprises an implant body having first and second configurations, the first configuration having reduced volume compared to the second configuration, the first configuration being dimensioned and configured to fit within a lumen of a surgical needle, the second configuration dimensioned and configured to fit between a superior articular face of a facet joint and an inferior articular face of the facet joint, the second configuration further dimensioned and configured to exert sufficient pressure against the superior articular face of the facet joint and the inferior articular face of the facet joint to keep said faces separated;
injecting the spinal facet implant into a facet joint; and
allowing the spinal facet implant to absorb bodily fluids and swell and seat the implant between the superior articular face of the facet joint and the inferior articular face of the facet joint.

22. An injectable facet joint cartilage replacement implant for treatment of a damaged facet joint comprising a fluid retaining hydrogel material dimensioned and configured for passage through a small diameter needle into a space within a facet joint, wherein upon absorption of fluid, the hydrogel material assumes a three dimensional conformation corresponding to the space between the facets to augment the function of damaged facet cartilage.

23. The injectable facet joint cartilage replacement implant according to claim 22 wherein the hydrogel material dimensioned and configured for passage through a small diameter needle is rod shaped.

24. The injectable facet joint cartilage replacement implant according to claim 22 wherein the three dimensional conformation corresponding to the space between the facets is disc shaped.

25. The injectable facet joint cartilage replacement implant according to claim 22 wherein the hydrogel material assumes the three dimensional conformation by absorption of bodily fluid.

26. The injectable facet joint cartilage replacement implant according to claim 22 wherein the hydrogel swells upon absorption of fluid and compresses by expression of fluid based upon load applied within the facet joint.

27. The injectable facet joint cartilage replacement implant according to claim 22 further comprising a support member disposed within the hydrogel.

28. The injectable facet joint cartilage replacement implant according to claim 27 wherein the support member includes an exteriorly disposed portion extending out of the implant body, the exteriorly disposed portion being dimensioned and configured to be attached to surrounding tissue and/or bone.

29. The injectable facet joint cartilage replacement implant according to claim 22 wherein the hydrogel gains mass when the hydrogel assumes a three dimensional shape conforming to the space between the facets.

30. An implant for treating inflammation associated with an irritated facet joint comprising a fluid absorbing injectable hydrogel material dimensioned and configured for passage through a small diameter needle into the synovial or capsular tissue surrounding the facet joint that absorbs inflammatory agents in the facet joint.

31. The implant for treating inflammation associated with an irritated facet joint according to claim 30 wherein the inflammatory agents are selected from the group consisting of cytokines and complement system components.

32. A method for treating inflammation associated with an irritated facet joint comprising positioning an at least partially dehydrated fluid absorbing polymer in the facet joint and allowing the fluid absorbing polymer to absorb and sequester fluids containing inflammatory agents from within the facet joint, thus reducing exposure of the inflammatory agents to the irritated facet joint.
